(19) United States
(12) Patent Application Publication
(10) Pub. No.: US 2009/0280194 A1
(43) Pub. Date: Nov. 12, 2009

(54) USE OF NITROXYL (HNO) FOR THE TREATMENT OF CANCERS OVEREXPRESSING MAT-8

(76) Inventors: Jeffrey P. FROEHLICH, Baltimore, MD (US); Nazareno PAOLOCCI, Baltimore, MD (US)

Correspondence Address: FOLEY & LARDNER LLP, 975 PAGE MILL ROAD, PALO ALTO, CA, 94304, US

(21) Appl. No.: 12/116,565

(22) Filed: May 7, 2008

(51) Int. Cl. A61K 33/00; A61P 35/00

(52) U.S. Cl. 424/722

(57) ABSTRACT

Compositions and methods for treating hormone dependent or independent cancers overexpressing MAT-8 in a patient by administration of nitroxyl-donating compounds and treating patient suffering from cancers such as prostate cancer, breast cancer, pancreatic cancer, colon cancer and lung cancer.

### FIELD OF THE INVENTION

The present invention generally relates to methods and compositions using nitroxyl or nitroxyl-donating compounds for treating cancers.

### BACKGROUND

Nitroxyl (HNO) donated by Angeli's salt (AS) is a cardiac muscle inotrope in vivo and in vitro. Paolocci et al. (2003) Proc. Natl. Acad. Sci. U.S.A. 100:5537-5542 and Tocchetti et al. (2007) Circ. Res. 100:96-104. Its unique pharmacological properties make it a potentially useful drug for maintaining cardiac function in end-stage congestive heart failure (CHF) where β-adrenergic blockers are no longer therapeutically effective. Paolocci et al. (2003) Proc. Natl. Acad. Sci. U.S.A. 100:5537-5542. Nitroxyl (HNO) is the one-electron reduction product of nitric oxide (NO), an endogenous vaso-relaxant generated by L-arginine metabolism. Fukuto et al. (2005) Chem. Res. Toxicol. 18:790-801. Dissolving AS in water at neutral pH spontaneously generates nitroxyl at 37° C. with a half-life of ˜2.5 minutes, which fully decomposes to nitrite after several hours at 37° C. and exposure to light. Fukuto et al. (2005) Chem. Res. Toxicol. 18:790-801 and Miranda et al. (2005) J. Med. Chem. 48:8220-8228. At physiological pH, decomposition of Angeli's salt is generally accepted to be initiated by protonation of the dianion and to produce nitroxyl (HNO) and nitrite. Miranda et al. (2005) J. Med. Chem. 48(26):8220-8228. Studies with isolated ventricular cardiac myocytes show that the cardiac effects of nitroxyl persist long after the drug has decayed to nitrite, indicating that nitroxyl induces a cascade of chemical modification of muscle proteins. Tocchetti et al. (2007) Circ. Res. 100:96-104. The specific nature of these changes is unknown, although recent data points to modification of critical thiol (cysteine) residues in proteins controlling calcium (Ca²⁺) uptake and release in cardiac muscle. Tocchetti et al. (2007) Circ. Res. 100:96-104, Pagliaro et al. (2003) Free Radic. Biol. Med. 34(1):33-43, Demaster et al. (1998) Biochem. Pharmacol. 55:2007-2015 and Bartberger et al. (2001) Proc. Natl. Acad. Sci. USA 98:2194-2198.

Intracellular Ca²⁺ homeostasis plays an important role in neoplastic progression and the apoptotic resistance of cancer cells. Preston et al. (1997) Cancer Research 57:537-542 and Prevarskaya et al. (2004) Biochem. Biophy. Res. Comm. 322:1326-1335. The uncontrolled growth of cells as seen in cancer of the prostate and breast is associated with a disruption in this Ca²⁺ homeostasis. Crepin et al. (2007) Biochemical J. 401:49-55, Legrand et al. (2001) J. Biol. Chem. 276:47608-47614 and Morrison et al. (1995) J. Biol. Chem. 270:2176-2182. Prostate and breast cancer, particularly their hormone independent variants, are a major cause of death in the adult male and female populations. Lee et al. (2007) Arch. Biochem. Biophys. 464:19-27 and Vanoverberghe et al. (2004) Cell Death and Differ. 11: 321-330. The divergent etiology of the hormone dependent and independent variants of prostate and breast cancer makes treatment of these cancers rather different. The main treatment for hormone dependent prostate or breast cancer is the inhibition of cell growth through the suppressing of action or production of endogenous hormones in the patient either through chemical or surgical means, known in the art as hormone therapy. Kurebayashi (2007) Breast Cancer 14(2):200-214 and Arianayagam et al. (2007) Aust. Fam. Physician 36(9):737-739. However, hormone therapy has significant side effects. For example, in prostate cancer, hormone therapy can result in erectile dysfunction, hot flushes, bone thinning, diabetes and congestive heart failure. Smith (2006) Clin. Cancer Res. 12(20 PT 2):6315s-6319s, Katz (2005) Can. Fam. Physician 51:977-982, Adelson et al. (2005) Expert Opin Pharmacother. 6(7):1095-1106, Keating et al. (2006) J. Clin. Oncol. 24(27):4448-4456 and Harlan et al. (2001) J. Natl. Cancer Inst. 93(24):1864-1871. For the hormone independent variants, hormone therapy has no effect on neoplastic cell growth as the cells do not express receptors for the associated hormone. Lee et al. (2007) Arch. Biochem. Biophys. 464:19-27. Therefore, alternative treatment including neoadjuvent or adjuvant chemotherapy are used to treat patients with hormone independent variants, but these alternative treatments have limited efficacy. Arianayagam et al. (2007) Aust. Fam. Physician 36(9):737-739. Thus, there is a need for the development of cancer treatments which are effective for both hormone dependent and independent neoplastic cells and in particular, hormone independent neoplastic cells.

One common feature of both hormone dependent and independent neoplastic prostate, breast, pancreatic, and lung cells is the overexpression of the mammary tumor, 8 kDa protein (MAT-8). Grzmil et al. (2004) Inter. J. Oncol. 24:97-105, Morrison et al. (1995) J. Biol. Chem. 270(5):2176-2182, Sweadner et al. (2000) Genomics 68:41-56, Kayed (2006) Int. J. Cancer 118(1):43-54 and Geering (2006) Amer. J. Physiol Renal Physiol. 290:F241-F250. However, MAT-8 is not overexpressed in all cancers and has been found to be down-regulated in some species of human colorectal cancer. Okumura et al. (2006) Molecular Mechanism and Regulation in Cation Transport ATPases and Related Genetic Disease, Kyoto Research Park, Kyoto Japan; Abstract: 108-109. MAT-8 is a member of the family of FXYD regulatory proteins, which inhibits the plasma membrane Na⁺/K⁺ pump, resulting in partial collapse of the transmembrane Na⁺ gradient. Sweadner et al (2000) Genomics 68:41-56. This gradient is important in maintaining cell shape, cell volume and the intracellular composition of ions through its action on Na⁺ gradient-dependent ionic transport mechanisms. The activity of the plasma membrane Na⁺/Ca²⁺ exchanger (NCX) is dependent on the Na⁺ gradient, which controls both the extent and direction of Ca²⁺ transport. When the Na⁺ gradient is high (140 mM Na⁺ outside: 5-10 mM Na⁺ inside), NCX pumps Ca²⁺ out of the cell, maintaining low levels of Ca²⁺ in the cytoplasmic and endoplasmic reticulum (ER) compartments. Miyashita et al. (1997) Amer. J. Physiol. 272 (Heart Circ. Physiol. 41):H244-H255 and Miyashita et al. (1997) Korean J. Gerontol. 7:82-93. However, when the sodium gradient is reduced, as it is in the presence of MAT-8, Ca²⁺ can enter the cell via NCX and be taken up into the ER compartment by an ATP-dependent Ca²⁺ pump, SERCA2b. Prevarskaya et al. (2004) Biochem. Biophys. Res. Comm. 322:1326-1335 and Legrand et al. (2001) J. Biol. Chem. 276:47608-47614. A major consequence of loading of the ER compartment with Ca²⁺ is protection of the cell against apoptosis, a natural or programmed means of cell death. Kaufman (1999) Genes & Dev. 13:1211-1233, Ashkenazi et al. (1999) Curr. Opin. Cell Biol. 11:255-260 and Gross et al. (1999) Genes & Dev. 13:1899-1911. Apoptosis prevents the uncontrolled growth of cancer cells by activating specific intracellular proteases (caspases) and DNAases, which cause destruction of the cell. When Ca²⁺ is prevented from being pumped into the ER, which occurs when SERCA2b is inhibited by thapsigargin, ER Ca²⁺ levels fall and the tumor cells experience “ER stress,” which induces apoptosis. He et al. (2002) Oncogene 21:2623-2633.

### SUMMARY OF THE INVENTION

This invention provides nitroxyl-donating compounds and compositions thereof that are useful for treating a neoplastic cell or cancer overexpressing MAT-8 protein. In one embodiment, the invention provides a method for inhibiting MAT-8 protein function in a neoplastic cell overexpressing MAT-8 protein by administering to the cell an effective amount of a nitroxyl-donating compound. In a further aspect, the inhibition of MAT-8 protein function, relieves is the inhibition of Na⁺/K⁺ transport across a plasma membrane by a Na⁺/K⁺ pump caused by overexpression of MAT-8. In yet a further aspect, the neoplastic cell is a neoplastic prostate cell, a neoplastic breast cell, a neoplastic pancreatic cell, a neoplastic colon cell or a neoplastic lung cell. In still yet a further aspect, the neoplastic cell is hormone dependent or hormone independent. Such hormones include, but are not limited to, androgens, estrogen, progesterone or equivalents thereof.

In another embodiment, the invention provides methods for treating a neoplastic prostate cell overexpressing MAT-8 by administering an effective amount of a nitroxyl-donating compound to the cell. In a further aspect, the cell is hormone dependent or independent. In yet a further aspect, the hormones from which the neoplastic prostate cell is dependent or independent on for neoplastic proliferation are androgens.

In another embodiment, the invention provides methods for treating a neoplastic breast cell overexpressing MAT-8 by administering an effective amount of a nitroxyl-donating compound to the cell. In a further aspect, the cell is hormone dependent or independent. In yet a further aspect, the hormones from which the neoplastic breast cell is dependent or independent on for neoplastic proliferation are estrogen or progesterone.

In another embodiment, the invention provides methods for treating a patient suffering from a cancer overexpressing MAT-8 protein by administering a therapeutically effective amount of a nitroxyl-donating compound thereby inhibiting MAT-8 protein function and treating the patient. In one aspect, the cancer is prostate cancer, breast cancer, pancreatic cancer, colon cancer or lung cancer. In yet another aspect the cancer is susceptible to hormone therapy. In still yet another aspect the cancer is not susceptible to hormone therapy.

In another embodiment, the invention is a method for treating a patient suffering from prostate cancer overexpressing MAT-8 protein by administering a therapeutically effective amount of a nitroxyl-donating compound thereby inhibiting MAT-8 protein function and treating the patient. In one aspect the cancer is susceptible to hormone therapy. In another aspect the cancer is not susceptible to hormone therapy.

In another embodiment, the invention is a method for treating a patient suffering from breast cancer overexpressing MAT-8 protein by administering a therapeutically effective amount of a nitroxyl-donating compound thereby inhibiting MAT-8 protein function and treating the patient. In one aspect the cancer is susceptible to hormone therapy. In another aspect the cancer is not susceptible to hormone therapy.

The methods described herein preferably include the administration of an effective amount or therapeutically effective amount of a nitroxyl-donating compound. In one aspect of the invention, one or more nitroxyl-donating compounds are used to provide nitroxyl to inhibit MAT-8 function in a neoplastic cell overexpressing MAT-8. Any physiologically acceptable nitroxyl-donating compound can be used. Such compounds include, but are not limited to, Angeli's salts (AS), glyceryl trinitrate (GTN), diazeniumdiolates (NONOate), diethylamine/NONOate (DEA/NO), Piloty's acid (PhSO₂NHOH, i.e. N-hydroxybenzensulfonamide or benzenesulfohydroxamic acid), cyanamide, hydroxylamine, acyl nitroso compounds, or derivatives of each thereof. In one aspect of the invention, the nitroxyl-donating compound is Angeli's salt of the formula (Na₂N₂O₃) is used to donate nitroxyl.

In another embodiment of the present invention, the methods include incorporating the nitroxyl-donating compound or composition described herein, with a pharmaceutically acceptable carrier and administering a therapeutically effective amount of the nitroxyl-donating compound or composition and the pharmaceutically acceptable carrier to a patient.

### DESCRIPTION OF THE FIGURES

FIG. 1 shows a schematic depiction of nitroxyl induced anti-neoplastic action against neoplastic cells overexpressing MAT-8. Up arrows indicate an increase in ion concentration or physiological activity of the cell, whereas down arrows indicate a decrease.

FIG. 2 shows inhibition of hormone dependent and independent prostate tumor cells by Angeli's salt, a nitroxyl-donating compound. PC3 cells (filled circles) are hormone independent cells, whereas, LNCaP (opend circles) are hormone dependent cells. Y-axis indicates percentage of cell growth as measured by a modified MTT assay. X-axis in the concentration (mM) of Angeli's salt added every 24 hrs for a total of 72 hours.

FIG. 3 shows a schematic depiction of the cardiac sarcoplasmic reticulum calcium pump (SERCA2a) transport mechanism of action. Steps 6 and 8 are the rate limiting steps in the system.

FIG. 4 shows activation of E2P hydrolysis in canine heart sarcoplasmic reticulum calcium pump (SERCA2a) by Angeli's salt, a nitroxyl-donating compound. Y-axis indicates the level of E2P (nmol/mg), which undergoes hydrolysis in step 6 of FIG. 3. X-axis indicates time following addition of Angeli's salt (sec). The inset figure represents a normalized comparison of E2P hydrolysis in the presence (+AS) or absence (−AS) of Angeli's salt.

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, cell biology, biochemistry, chemistry, pharmacology and immunology, which are within the skill of the art. Such techniques are explained fully in the literature for example in the following publications. See, e.g., Sambrook and Russell eds. MOLECULAR CLONING: A LABORATORY MANUAL, 3rd edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); CULTURE OF ANIMAL CELLS: A MANUAL OF BASIC TECHNIQUE (R. I. Freshney 5^{th} edition (2005)); and GOOMAN AND GILLMAN'S THE PHARMACOLOGICAL BASIS OF THERAPEUTICS (Brunton et al. McGraw Hill Publishing (2005)).

### DEFINITIONS

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

“Nitroxyl” (HNO/NO—) or hyponitrous acid, is the reduced form of nitric oxide (NO). HNO and NO— form an acid/base pair (pKa=11.4) with NO— isoelectronic with dioxygen. Nitroxyl is very reactive towards nucleophiles (especially thiols) and quickly dimerizes to H₂N₂O₂, which then is dehydrated to nitrous oxide N₂O. Therefore, HNO is generally prepared in situ for example with the compounds such as Angeli's salts (AS), glyceryl trinitrate (GTN), diazeniumdiolates (NONOate), diethylamine/NONOate (DEA/NO), Piloty's acid (PhSO₂NHOH, i.e. N-hydroxybenzensulfonamide or benzenesulfohydroxamic acid), cyanomide, hydroxylamine, acyl nitroso compounds, or derivatives of each thereof.

“Mammary Tumor 8 kDa protein” or “MAT-8” (also known as FXYD domain-containing ion transport regulator 3 (FXYD3)) is a member of the family of FXYD regulatory proteins, which inhibits the plasma membrane Na⁺/K⁺ pump, resulting in partial collapse of the transmembrane Na⁺ gradient. MAT-8 was first identified as a novel 8-kDa transmembrane protein in mouse that was expressed in a subset of mouse breast tumors initiated by neu or ras oncogenes. Morrison et al. (1994) Oncogene 9:3417-3426. The human MAT-8 cDNA encodes a deduced 67 amino acid protein with a calculated mass of 8 kDa and shows partial homology to phospholemman (PLM). It shares homologous extracellular and transmembrane domains with PLM, but has 2 cysteine residues in the transmembrane domain separated by 4 amino acids. This protein have been sequenced and characterized, see for example GenBank Accession Nos. NP_{—}005962.1 and NP_{—}068710.1. The gene for this protein has also been sequenced and characterized, see for example GenBank Accession Nos. NM_{—}005971.2 and NM_{—}021910.1.

The phrase “hormone therapy” refers to treatment that adds, blocks, or removes hormones. For example, to slow or stop the growth of certain cancers, such as but not limited to prostate or breast cancer, synthetic hormones or other drugs may be given to block the body's natural hormones. In another aspect, surgery is used to remove the gland that makes the hormone. “Hormone therapy” is also known in the art as hormonal therapy, hormone treatment or endocrine therapy. As described herein, when a cancer cell or tumor is “susceptible” to hormone therapy, this refers to the inhibition of cell proliferation and/or cell death of the corresponding cancer cell or tumor being treated as a result of the administration of hormone therapy. Conversely, when a cancer cell or tumor is “not susceptible” to hormone therapy, the cell or tumor proliferation and/or cell death is not inhibited by the administration of hormone therapy.

“Expression” as applied to a gene, refers to the differential production of the mRNA transcribed from the gene or the protein product encoded by the gene. A differentially expressed gene may be overexpressed (high expression) or underexpressed (low expression) as compared to the expression level of a normal or control cell, a given patient population or with an internal control. In one aspect, it refers to a differential that is about 1.5 times, or alternatively, about 2.0 times, alternatively, about 2.0 times, alternatively, about 3.0 times, or alternatively, about 5 times, or alternatively, about 10 times, alternatively about 50 times, or yet further alternatively more than about 100 times higher or lower than the expression level detected in a control sample. The term “expressed” also refers to nucleotide sequences or polypeptides in a cell or tissue which are expressed where silent in a control cell or not expressed where expressed in a control cell. In another aspect, expression level is determined by measuring the expression level of a gene of interest for a given patient population, determining the median expression level of that gene for the population, and comparing the expression level of the same gene for a single patient to the median expression level for the given patient population. For example, if the expression level of a gene of interest for the single patient is determined to be above the median expression level of the patient population, that patient is determined to have high expression of the gene of interest. Alternatively, if the expression level of a gene of interest for the single patient is determined to be below the median expression level of the patient population, that patient is determined to have low expression of the gene of interest.

As used herein, the term “gene of interest” intends the gene which expresses the MAT-8 protein.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “hormone” refers to all naturally occurring and synthetic compounds which facilitate the neoplastic growth of cells or proliferation of cancer cells of the prostate, breast, pancreas, colon or lung, including metastizing of a cell from a primary tumor to another tissue. Examples of such hormones include, but are not limited to, androgens (i.e. testosterone, dehydroepiandrosterone (DHEA), androstenedione, androstenediol, androsterone or dihydrotestosterone), estrogens (i.e. estriol, estradiol, estrone, equilin or equilenin, for other examples see, Fang et al. (2001) Chem. Res. Toxicol. 14(3):280-294) and progesterones (i.e. progesterone, pregnenolone and dioxycorticosterone). Additional examples of androgens, estrogens, and progesterones can be found at the National Center for Biotechnology Information within the PubChem Compound, PubChem Substance, PubChem BioAssay and Structure Search databases at www.ncbi.nlm.nih.gov, last visited on May 4, 2008.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically-acceptable carrier” encompasses any of the standard pharmaceutical carriers, such as a phosphate-buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

An “excipient” refers to an inert substance added to a pharmaceutical composition to further facilitate administration of the active ingredient.

A “subject,” “individual” or “patient” is used interchangeably herein, and refers to a vertebrate, for example a mammal or preferably a human. Mammals include, but are not limited to, murines, rats, simians, humans, farm animals, sport animals and pets.

An “effective amount” is used synonymously with a “therapeutically effective amount” and intends an amount sufficient to effect beneficial or desired results. An effective amount can be administered in one or more administrations, applications, or dosages.

“Treating” or “treatment” of a disease, disorder or condition will depend on the disease, disorder or condition to be treated and the individual to be treated. In general, treatment intends one or more of (1) inhibiting the progression of the manifested disease, disorder or condition as measured by clinical or sub-clinical parameters (where the term “Inhibiting” or “Inhibition” is intended to be a subset of “Treating” or “treatment”), (2) arresting the development of the disease, disorder or condition as measured by clinical or sub-clinical parameters, (3) ameliorating or causing regression of the disease, disorder or condition as measured by clinical or sub-clinical parameters, or (4) reducing pain or discomfort for the subject as measured by clinical parameters. For example, in the case of cancer, a response to treatment includes a reduction in cachexia, increase in survival time, elongation in time to tumor progression, reduction in tumor mass, reduction in tumor burden and/or a prolongation in time to tumor metastasis, each as measured by standards set by the National Cancer Institute and the U.S. Food and Drug Administration for the approval of new drugs. See Johnson et al. (2003) J. Clin. Oncol. 21(7): 1404-1411. “Treating” does not include preventing the onset of the disease or condition.

“Preventing” or “prevention” of a disease, disorder or condition means that the onset of the disease or condition in a subject predisposed thereto is prevented such that subject does not manifest the disease, disorder or condition.

### DESCRIPTIVE EMBODIMENTS

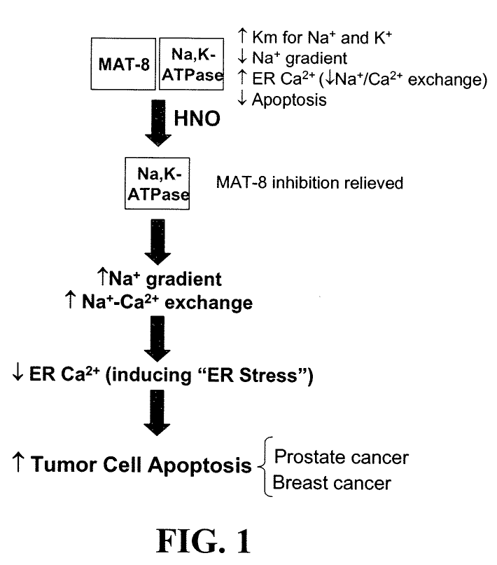

Without being bound by theory, a mechanism for nitroxyl action in tumor cells overexpressing MAT-8 is shown in FIG. 1. Intravenous administration of the water-soluble drug and protonation of nitroxyl anion facilitate the contact of nitroxyl with MAT-8 in the plasma cell membrane, resulting in chemical modification (disulfide bond formation) of transmembrane cysteine residues. The subsequent conformational change in MAT-8 will weaken its association with the sodium pump, relieving its inhibition and increasing Na⁺ efflux from the cell. This will increase the transmembrane sodium gradient, the driving force for sodium-calcium exchange, increasing Ca²⁺ efflux from the cell, thereby reducing the cytoplasmic and ER Ca²⁺ levels. The drop in ER Ca²⁺ content, which results from the depletion of cytoplasmic Ca²⁺ and leakage of Ca²⁺ (via Ca²⁺-selective channels) out of the ER, triggers ER stress, rendering the cell susceptible to apoptosis which causes its destruction. Because MAT-8 is overexpressed in hormone independent as well as in hormone dependent prostate and breast tumors, controlling the growth and spread of both cell types is done by administration of a nitroxyl-donating compound. The primary advantage of this approach over one that induces ER stress by inhibiting ER Ca²⁺ uptake by SERCA2b (e.g., with thapsigargin), which is widely distributed in the body, is that only those cells overexpressing MAT-8 (i.e. select tumor cells) are targeted by nitroxyl, enhancing its selectivity and reducing its cytotoxicity. The advantage the tumor cell has in preventing ER stress by up-regulating MAT-8 and inhibiting the sodium pump is thus nullified, making the tumor cells susceptible to apoptosis, the natural body defense mechanism inherently present in all cells for preventing their uncontrolled proliferation and metastasis.

### Therapeutic Methods

The present invention is directed to the use of nitroxyl-donating compounds to treat a neoplastic cell or cancer overexpressing MAT-8 protein or inhibit MAT-8 function in a neoplastic cell overexpressing MAT-8. The present invention is further directed to the surprising and unexpected discovery that use of nitroxyl-donating compounds described herein can inhibit the growth or proliferation of hormone dependent and hormone independent neoplastic cells or cancers. Hormone dependent and hormone independent neoplastic cells include, but not limited to, a neoplastic prostate cell, a neoplastic breast cell, a neoplastic pancreatic cell, a neoplastic colon cell and a neoplastic lung cell.

It has previously been shown that nitroxyl-donating compounds can inhibit cyclooxygenase-2 activity, maintaining cardiac function in end-stage congestive heart failure and act as phosphodiesterase inhibitors, see e.g. U.S. Pat. Nos. 6,936,639; 6,930,113; 6,462,044; 6,331,543; 6,469,065 and U.S. Publication Nos.: 2005/0009789. However, it was not known prior to the present invention that nitroxyl-donating compounds described herein can selectively inhibit neoplastic cell proliferation of MAT-8 overexpressing neoplastic cells irrespective of hormone dependence.

“Prostate cancer” is a disease in which a neoplastic prostate cell develops into a malignant tumor in the prostate, a gland in the male reproductive system. Neoplastic prostate cells are known to metastasize to other parts of the body, especially to the bones and lymph nodes. Prostate cancer generically includes both hormone dependent and independent neoplastic cells.

“Breast cancer” is a disease in which a neoplastic breast cell develops into a malignant tumor in the breast. The world health organization (WHO) classifies the tumors of the breast into a number of pathological types (i.e. invasive breast carcinomas, mesenchymal tumors, precursor lesions, benigh epithelial lesions, myoepithelial lesions, fibroepithelial tumors, tumors of the nipple, malignant lymphoma and metastatic tumors), see “Pathology and Genetics of Tumours of the Breast and Female Genital Organs” eds. Tavassoli and Devillee (2003). Breast cancer generically includes both hormone dependent and independent neoplastic cells.

Accordingly, in one aspect the invention provides a method for inhibiting MAT-8 protein function in a neoplastic cell overexpressing MAT-8 protein comprising administering to the cell an effective amount of a nitroxyl-donating compound. In a further aspect, the MAT-8 protein function is the inhibition of Na⁺/K⁺ transport across a plasma membrane by a Na⁺/K⁺ pump. In yet a further aspect, the nitroxyl-donating compound is Angeli's salt of the formula (Na₂N₂O₃). In yet a further aspect, the neoplastic cell is a neoplastic prostate cell, a neoplastic breast cell, a neoplastic pancreatic cell, a neoplastic colon cell or a neoplastic lung cell.

In a further aspect of this invention, the neoplastic cells of the methods above are hormone dependent or hormone independent. Such hormones include, but are not limited to, androgens, estrogen, progesterone or equivalents thereof. Thus, in a further aspect, the neoplastic prostate cell is androgen dependent. In another aspect the neoplastic prostate cell is androgen independent. In yet another aspect, the neoplastic breast cell is estrogen or progesterone dependent. In still yet another aspect, the neoplastic breast cell is estrogen or progesterone independent.

In another embodiment, the invention provides methods for treating a neoplastic prostate cell overexpressing MAT-8 comprising administering an effective amount of a nitroxyl-donating compound to the cell. In a further aspect, the cell is hormone dependent or independent. In yet a further aspect, the hormones from which the neoplastic prostate cell is dependent or independent on for neoplastic proliferation are androgens. In yet a further aspect, the nitroxyl-donating compound is Angeli's salt of the formula (Na₂N₂O₃).

In another embodiment, the invention provides methods for treating a neoplastic breast cell overexpressing MAT-8 comprising administering an effective amount of a nitroxyl-donating compound to the cell. In a further aspect, the cell is hormone dependent or independent. In yet a further aspect, the hormones from which the neoplastic breast cell is dependent or independent on for neoplastic proliferation are estrogen or progesterone. In yet a further aspect, the nitroxyl-donating compound is Angeli's salt of the formula (Na₂N₂O₃).

In another embodiment, the invention provides methods for treating a patient suffering from a cancer overexpressing MAT-8 protein that comprises administering a therapeutically effective amount of a nitroxyl-donating compound thereby inhibiting MAT-8 protein function and treating the patient. In one aspect, the cancer is prostate cancer, breast cancer, pancreatic cancer, colon cancer or lung cancer. In yet another aspect the cancer is susceptible to hormone therapy. In still yet another aspect the cancer is not susceptible to hormone therapy. In still yet a further aspect, the nitroxyl-donating compound is Angeli's salt of the formula (Na₂N₂O₃).

In another embodiment, the invention is a method for treating a patient suffering from prostate cancer overexpressing MAT-8 protein that comprises administering a therapeutically effective amount of a nitroxyl-donating compound thereby inhibiting MAT-8 protein function and treating the patient. In one aspect the cancer is susceptible to hormone therapy. In another aspect the cancer is not susceptible to hormone therapy. In yet a further aspect, the nitroxyl-donating compound is Angeli's salt of the formula (Na₂N₂O₃).

In another embodiment, the invention is a method for treating a patient suffering from breast cancer overexpressing MAT-8 protein that comprises administering a therapeutically effective amount of a nitroxyl-donating compound thereby inhibiting MAT-8 protein function and treating the patient. In one aspect the cancer is susceptible to hormone therapy. In another aspect the cancer is not susceptible to hormone therapy. In yet a further aspect, the nitroxyl-donating compound is Angeli's salt of the formula (Na₂N₂O₃).

### Nitroxyl and Nitroxyl-Donating Compounds

In each of the above embodiments, an effective amount of a nitroxyl-donating compound, or composition comprising a nitroxyl-donating compound, is administered to a subject in need thereof. One of ordinary skill in the art would be able to determine these and other compounds capable of donating nitroxyl. Also included in this invention is direct administration of nitroxyl to a cell or patient in need thereof as described herein.

A nitroxyl donor is an agent or compound that provides a physiologically effective amount of nitroxyl (HNO). The nitroxyl-donating compound is any compound that donates HNO and has a safety profile indicating the compound would be tolerated by a subject in the amount necessary to achieve a therapeutic effect. The mechanism of nitroxyl donation will vary depending on the compound. One non-limiting example of such a mechanism is shown in reaction of Scheme 1, wherein Angeli's salt of the formula Na₂N₂O₃ is dissolved in water at a pH of 7.4, therein releasing nitroxyl. Examples of other nitroxyl donating compounds useful for this invention include, but are not limited to, glyceryl trinitrate (GTN), diazeniumdiolates (NONOate), diethylamine/NONOate (DEA/NO), Piloty's acid (PhSO₂NHOH, i.e. N-hydroxybenzensulfonamide or benzenesulfohydroxamic acid), cyanamide, hydroxylamine, acyl nitroso compounds, or derivatives of each thereof, see also Miranda et al. (2005) Curr. Top. Med. Chem. 5(7):649-664, herein incorporated by reference in it entirety.

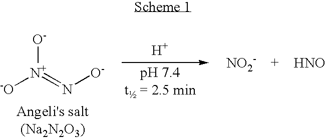

Additionally, analogs and derivatives of such compounds can be used. Moreover, conditions, such as the oxidation state of the environment, can be altered to cause such compounds to donate nitroxyl.

In another aspect of the invention, one or more of the nitroxyl-donating compounds, may be used in the preparation of a medicament for the treatment of a patient suffering from a cancer overexpressing MAT-8 protein including, but not limited to prostate cancer, breast cancer, pancreatic cancer, colon cancer and lung cancer.

### Compositions and Formulations

The compositions are comprised of, in general, a nitroxyl-donating compound in combination with at least one pharmaceutically acceptable carrier or excipient. Acceptable carriers are known in the art and described supra. Acceptable excipients are non-toxic, aid administration, and do not adversely affect the therapeutic benefit of the compound. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols.

The nitroxyl-donating compound can be administered in any suitable formulation such as a tablet, pill, capsule, semisolid, gel, transdermal patch or solution, powders, sustained release formulation, solution, suspension, elixir or aerosol. The most suitable formulation will be determined by the disease or disorder to be treated and the individual to be treated.

Compressed gases may be used to disperse a nitroxyl-donating compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The following are representative pharmaceutical formulations containing a nitroxyl-donating compound of the present invention.

### Tablet Formulation

The following ingredients are mixed intimately and pressed into single scored tablets.

| Ingredient | Quantity per tablet, mg |
|---|---|
| Nitroxyl-donating compound | 400 |
| Cornstarch | 50 |
| Croscarmellose sodium | 25 |
| Lactose | 120 |
| Magnesium stearate | 5 |

### Capsule Formulation

The following ingredients are mixed intimately and loaded into a hard-shell gelatin capsule.

| Ingredient | Quantity per capsule, mg |
|---|---|
| Nitroxyl-donating compound | 200 |
| Lactose, spray-dried | 148 |
| Magnesium stearate | 2 |

### Suspension Formulation

The following ingredients are mixed to form a suspension for oral administration (q.s.=sufficient amount).

| Ingredient | Amount | |
|---|---|---|
| Nitroxyl-donating compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.0 | g |
| Veegum K (Vanderbilt Co) | 1.0 | g |
| Flavoring | 0.035 | mL |
| colorings | 0.5 | mg |
| distilled water | q.s. to 100 | mL |

### Injectable Formulation

The following ingredients are mixed to form an injectable formulation.

| | Quantity per injection, |
|---|---|
| Ingredient | mg |
| Nitroxyl-donating compound | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| | Quantity per |
|---|---|
| Ingredient | suppository, mg |
| Nitroxyl-donating compound | 500 mg |
| Witepsol ® H-15 | balance |

Also provided is a medicament comprising a compound or composition as described herein for use in treating a disease or disorder as described above, which can be identified by noting any one or more clinical or sub-clinical parameters.

### Combination Therapy

For more generalized therapeutic purposes, combination therapy is often desirable. Combination therapy includes administration of a single pharmaceutical dosage formulation which contains a nitroxyl-donating compound and one or more additional active agents, or therapies such as heat, light and such, as well as administration of the nitroxyl-donating compound and each active agent in its own separate pharmaceutical dosage formulation. For example, a compound of this invention and one or more of other agents including, but are not limited to, compounds for hormone therapy such as steroids, finasteride, aromatase inhibitors, gonadotropin-releasing hormone agonist (i.e. goserolin), hormone receptor antagonist (i.e. antiandrogens such as flutamide and bicalutamide, selective estrogen receptor modulators (SERM's) such as tamoxifen, faloxifene, toremifene and fulvestrant), hormone supplementation (i.e. progestagens, androgens, estrogens and somatostatin analogs) and hormonal immunotherapy (i.e. interferons and cytokines) could be administered to the human subject together in a single dosage composition such as a tablet, capsule, injection or each agent can be administered in separate dosage formulations. Furthermore, non-medical hormonal interventions can be combined with the administration of one or more of the herein described compounds including removal or destruction of endocrine organs through surgery (i.e. castration or removal of ovaries in females) or radiation therapy. Other useful agents in the treatment of the cancers overexpressing MAT-8 include chemotherapeutic agents, such as but not limited to, alkylating agents, anti-metabolites, plant alkaloids and terpenoids, vinca alkaloids, podophyllotoxin, taxanes, topoisomerase inhibitors, antitumour antibodies and monoclonal antibodies. Combination therapy is understood to include all these regimens.

### Dosing and Administration

The present invention provides therapeutic methods generally involving administering to a subject in need thereof an effective amount of nitroxyl-donating compound described herein. The dose, frequency, and timing of such administering will depend in large part on the selected therapeutic agent, the nature of the condition to be treated, the condition of the subject, including age, weight and presence of other conditions or disorders, the formulation of the therapeutic agent and the discretion of the attending physician. The nitroxyl-donating compounds and compositions described herein and the pharmaceutically acceptable salts thereof are administered via oral, parenteral, subcutaneous, intramuscular, intravenous or topical routes. In particular embodiments, the nitroxyl-donating compound is administered by a short-term intravenous infusion, such as for 5 to 20 minutes. In other embodiments the nitroxyl-donating compound is administered by a long-term intravenous infusion, such as from 3-4 hours. In yet other embodiments, the nitroxyl-donating compound is administered as a bolus intravenous infusion/injection. Generally, it is contemplated that the nitroxyl-donating compounds are to be administered in dosages ranging from about 0.10 milligrams (mg) up to about 1000 mg per day, although variations will necessarily occur, depending, as noted above, on the target tissue, the subject, and the route of administration. In particular embodiments, an infusion of nitroxyl-donating compound of 10 micrograms (μg)/kilogram of body weight (kg)/minute (min) is administered for 5-20 min. In one example, the nitroxyl-donating compound administered at this dose is Angeli's salt. In other embodiments an infusion of 25 μg/kg/min is administered for 5-20 min.

The following examples are provided to illustrate certain aspects of the present invention and to aid those of skill in the art in practicing the invention. These examples are in no way to be considered to limit the scope of the invention.

### EXAMPLES

The examples below as well as throughout the application, the following abbreviations have the following meanings. If not defined, the terms have their generally accepted meanings.

DMSO=dimethylsulfoxide

EGTA=ethylene glycol tetraacetic acid

SBFI=sodium-binding benzofuran isophthalate

g=gram

m=multiplet

M=molar

mg=milligram

mL=milliliter

dL=deciliter

mM=millimolar

mmol=millimole

μL=microliter

### Example 1

Growth Inhibition of Androgen Dependent and Independent Prostate Tumor Cells

A characteristic feature of PC3 (androgen independent) and LNCaP (androgen dependent) prostate tumor cells is the overexpression of an intrinsic plasma membrane Na⁺/K⁺ pump regulatory protein, MAT-8. As described above, overexpression of MAT-8 is a property shared with breast, pancreatic and lung carcinomas. The growth inhibition of PC3 and LNCaP cells by nitroxyl (HNO) donated by Angeli's salt (AS) was investigated.

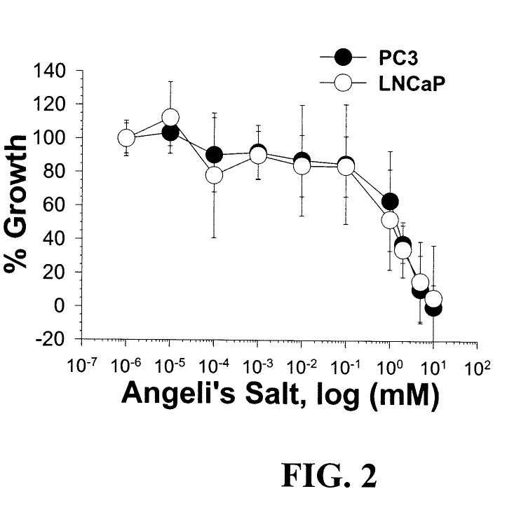

PC3 (2500 cells/well) and LNCaP (4000 cells/well) cells were cultured in 200 μl of incubation media in each well of a 96-well assay plate 24 hours prior to the addition of AS. Following an overnight incubation at 37° C., appropriate AS concentrations were added to each well from a fresh stock solution of 0.5 M AS (Na₂N₂O₃) in 10 mM NaOH (added to stabilize AS) diluted to 30 mM with cell culture medium. AS/HNO was added every 24 hours for a total of 72 hours. Cell viability was assayed using the MTT assay, a colorimetric method performed with minor modification according to the procedure of Mossman (1983) J. Immunol. Methods 65:55-63. Both PC3 and LNCaP cells showed growth inhibition by 50% (EC₅₀) at a concentration of 1.2 to 1.4 mM of Angeli's salt (FIG. 2). Over a series of three separate experiments, nitroxyl generated from AS inhibited the growth of cultured prostate PC3 and LNCaP cells with an EC₅₀ of 1.0-1.7 mM (Table 1). Control experiments, in which NaOH was added to the cells at a dose equal to that introduced with AS in the stock solution, showed no effect on cell growth or viability. Because AS is a highly water soluble drug and rapidly permeates cells (Paolocci et al. (2003) Proc. Natl. Acad. Sci. U.S.A. 100:5537-5542 and Tocchetti et al. (2007) Circ. Res. 100:96-104), the use of DMSO or a similar vehicle to enhance its access to cells and cell membranes was unnecessary. These results, in combination with the herein described examples, demonstrates that nitroxyl-donating compounds can be used to treat cancers overexpressing MAT-8, such as, but not limited to, prostate cancer.

TABLE 1
EC₅₀ values (mean, mM) to Angeli's Salt (AS) from 3 independent
experiments*

| | Experiment 1 | Experiment 2 | Experiment 3 | |
|---|---|---|---|---|
| Cell Line | (EC₅₀) | (EC₅₀) | (EC₅₀) | Mean |
| PC3 | 1.4 | 0.7 | 1.4 | 1.17 |
| LNCaP | 3.0 | 0.9 | 1.2 | 1.70 |
| LNCaP | — | 0.9 | 1.2 | 1.05 |

*EC₅₀ = AS concentration at which 50% growth inhibition occurs

### Example 2

### Nitroxyl (HNO) Activation of the Cardiac Muscle Sarcoplasmic Reticulum Ca²⁺ Pump (SERCA2a) Isolated from Canine Heart Cells

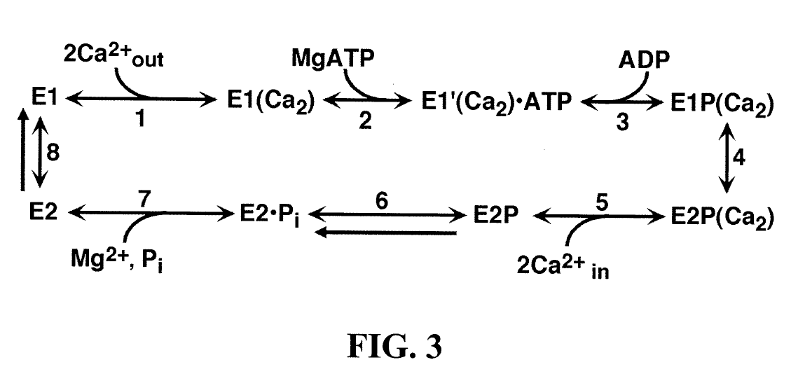

Investigations of the mechanism of the cardiac muscle sarcoplasmic reticulum (SR) Ca²⁺ pump (SERCA2a) have provided a paradigm for understanding how the interaction of nitroxyl with a regulatory protein (phospholamban) can activate the pump. SERCA2a is a P-type cation pump, i.e., it utilizes ATP as its energy source for uphill Ca²⁺ transport and becomes phosphorylated by ATP during the catalytic cycle (FIG. 3). Regulation of its activity by the small intrinsic cardiac SR membrane protein, phospholamban (PLB), involves primarily steps 6 and 8 which are rate-limiting in the overall transport cycle and control Vmax (steps 6 and 8) and the affinity of the cytoplasmic Ca²⁺ transport sites, Km (step 8). Mahaney et al. (2005) Biochemistry 44:7713-7724 and Antipenko et al. (1997) Biochemistry 36:12903-12910. PLB typically acts as an inhibitor of SERCA2a transport, and this inhibition is relieved upon phosphorylation of PLB. The majority of studies of the basic transport mechanism and its regulation by PLB have been carried out using sealed vesicles of SR isolated from cardiac muscle by differential centrifugation. Using SR vesicles prepared from mouse heart, previous studies have shown that brief exposure to AS (0.25 mM) activates Ca²⁺uptake measured by stopped-flow mixing, increasing the rate (efficiency) of Ca²⁺ uptake rather than the total amount of uptake. Tocchetti et al. (2007) Circ. Res. 100:96-104. This effect of AS/HNO can be explained by an increase in the activity of rate-limiting steps in the Ca²⁺ transport mechanism (steps 6 & 8 in FIG. 3) since these reactions control the overall cycling rate and hence the Ca²⁺ uptake activity of the SR Ca²⁺ pump. To investigate whether E2P hydrolysis (step 6) is activated by AS/HNO, its effect on EGTA-induced dephosphorylation of SERCA2a in native cardiac SR vesicles isolated from dog heart was assayed.

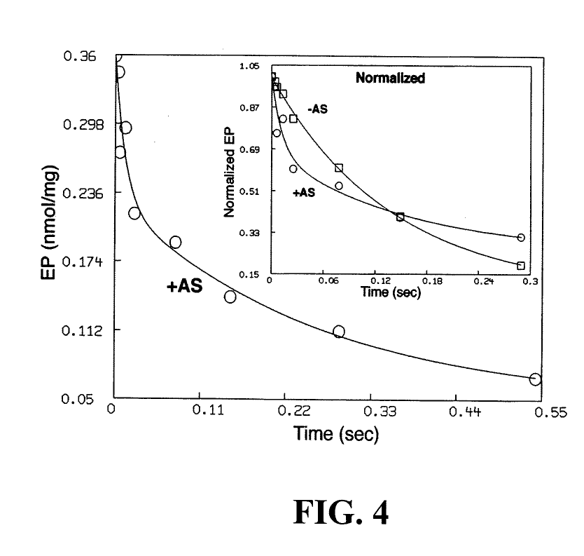

SERCA2a was initially phosphorylated at the catalytic site using 10 μM [γ³²P]ATP and then subsequently dephosphorylated by chasing with 5 mM EGTA. EGTA chelates the Ca²⁺ necessary for activation of catalytic site phosphorylation, preventing re-phosphorylation of the pump and exposing spontaneous hydrolysis of the phosphoenzyme intermediate, E2P (FIG. 3). In the absence of AS/HNO, E2P decay measured at 24° C. by quenched-flow mixing in dog cardiac SR is slow (11 s⁻¹) and monophasic (FIG. 4). However, exposure to 0.25 mM AS induces a biphasic decay pattern with an accelerated initial decay phase (77 s⁻¹) (FIG. 4), indicating that the hydrolysis of E2P (FIG. 3, step 6) is activated by AS/HNO.

### Example 3

### Nitroxyl (HNO) Activates the Cardiac Muscle Sarcoplasmic Reticulum Ca²⁺ Pump (SERCA2a) by Reacting with Proximal Thiols in the Transmembrane Domain of Phospholamban

Nitroxyl (HNO) donated by Angeli's salt (AS) enhances intracellular Ca²⁺ cycling in isolated cardiac myocytes by activating Ca²⁺ release from ryanodine (RyR2) receptors and Ca²⁺ re-uptake by the SR Ca²⁺ pump. Tocchetti et al. (2007) Circ. Res. 100:96-104. In cardiac SR vesicles, activation of Ca²⁺ uptake by HNO involves a direct interaction with the pump and is accompanied by enhanced dephosphorylation of the cardiac Ca²⁺-ATPase isozyme, SERCA2a. To determine whether HNO interacts with SERCA2a or its regulatory protein, phospholamban (PLB) and to characterize the protein site(s) modified by HNO, activation of SERCA2a dephosphorylation by nitroxyl using SERCA2a and PLB (WT and mutant) expressed in Sf21 cell ER microsomes (baculovirus system) was investigated. Brief exposure of SERCA2a+WT PLB to 0.25 mM AS produced a 10-fold stimulation of dephosphorylation (0.5 s⁻¹→5 s⁻¹) following an EGTA chase (Table 2). No additional stimulation was observed in microsomes expressing SERCA2a alone, demonstrating that PLB is necessary for the nitroxyl effect (Table 2). SERCA2a co-expressed with mutant PLB (—C) in which all 3 cysteine residues in the transmembrane domain were replaced by alanine (Cys→Ala) showed no stimulation by nitroxyl (Table 2). Exposure to an anti-PLB antibody resulted in a decrease in Km for Ca²⁺ uptake and showed that regulation of SERCA2a by PLB (—C) remained intact. Exposure to dithiothreitol (2 mM DTT) reversed the activation of dephosphorylation by nitroxyl, similar to its effect on HNO-induced activation of Ca²⁺ release from the RyR2 receptor. Tocchetti et al. (2007) Circ. Res. 100:96-104. These results demonstrate that activation of the cardiac SR Ca²⁺ pump by nitroxyl is phospholamban-dependent and results from covalent modification of its cysteine residues. Reversal of this effect by DTT suggests 1) that activation by nitroxyl involves disulfide bond formation between proximal thiols in the alpha-helical transmembrane domain of PLB, and 2) that conformational strain in PLB introduced by this bond weakens its interaction with SERCA2a and relieves its inhibition.

TABLE 2
Effect of AS/HNO on E2P hydrolysis in Sf21 ER microsomes
at 0° C.*

| | AS | | | | |
|---|---|---|---|---|---|
| Preparation | (mM) | A₁ (%) | k₁ (s⁻¹) | A₂ (%) | k₂ (s⁻¹) |
| SERCA2a + PLB (WT) | 0 | 77 | 0.47 | 23 | 0.09 |
| SERCA2a + PLB (WT) | 0.25 | 11 | 5.00 | 89 | 0.154 |
| SERCA2a alone | 0 | 21 | 4.34 | 79 | 0.233 |
| SERCA2a alone | 0.25 | 23 | 4.64 | 77 | 0.174 |
| SERCA2a + PLB (−C) | 0 | 100 | 0.16 | — | — |
| SERCA2a + PLB (−C) | 0.25 | 100 | 0.18 | — | — |

*A₁ = amplitude fast phase; A₂ = amplitude slow phase. A₁ + A₂ = 100%.

Thus, in the absence of PLB, SERCA2a is already in an activated state, and taking together Examples 2 and 3, these results suggest that AS/HNO activates E2P hydrolysis and SR Ca²⁺ uptake by relieving the inhibition imposed by PLB. Activation of these transport reactions presumably involves the covalent modification of PLB by HNO since that could change its conformational state and weaken its interaction with SERCA2a, allowing Ca²⁺ pumping activity to increase.

### Example 4

### Nitroxyl Induced Apoptosis in MAT-8 Overexpressing Prostate and Breast Cancer Cells

Human prostate cancer cell lines (DU145, PC-3BM, TSU-Pr1, and ALVA31), human breast cancer cell lines (MCF-7, T47D) and cells grown in primary culture derived from fresh surgical specimens of these tissues including normal prostate and breast tissue are isolated and cultured using standard cell culture techniques as described in CULTURE OF ANIMAL CELLS: A MANUAL OF BASIC TECHNIQUE (R. I. Freshney 5^{th} edition (2005)). Overexpression of MAT-8 is tested in extracts from the above cells by quantitative Western blotting using a polyclonal antibody to human MAT-8 as described in Sambrook and Russell eds. MOLECULAR CLONING: A LABORATORY MANUAL, 3rd edition (2001). Tumor cells overexpressing MAT-8 are exposed to varying concentrations of Angeli's salt (AS). The modified MTT assay as described in Mossman (1983) J. Immunol. Methods 65:55-63 is used to determine the dose-response relationship and EC₅₀ for growth inhibition. To assay for nitroxyl-induced Na⁺/K⁺ pump activation in cell lines demonstrating growth inhibition by AS, cells are loaded with the Na⁺-sensitive fluorescent dye, SBFI, and the cytoplasmic Na⁺ concentration is monitored by fluorescence microscopy prior to exposure with AS, obtaining a baseline Na⁺ fluorescence, and for several hours after exposure to an EC₅₀ dose of AS as described in Levi et al. (1994) J. Cardiovasc. Electrophysiol. 5(3):241-257. Activation of the Na⁺/K⁺ pump is detected as a fall in the cytoplasmic Na⁺ concentration from the baseline levels in which MAT-8 inhibition is present. A separate population of cells is loaded with Mg-fluo-4, a Ca²⁺-sensitive dye that localizes to the ER and reports on the ER free Ca²⁺ concentration as described in Criddle et al. (2006) Gastroenterology 130(3):781-793. Measurements of Mg-fluo-4 fluorescence by confocal fluorescence microscopy is made before and after exposure to AS and correlated with drug-induced changes in cytoplasmic Na⁺. A decrease in the ER Ca²⁺ content observed in unison with a drop in cytoplasmic Na⁺ following exposure to AS signals activation of Ca²⁺ efflux via the Na⁺/Ca²⁺ exchanger in the plasma cell membrane. These molecular events occur in advance of any biochemical and morphological changes indicating the onset of apoptosis. The morphological presence of apoptosis is analyzed by the detection of chromatin condensation and nuclear fragmentation in 300-1000 nuclei contained in 5-10 randomly-selected fields as described in Lee et al. (2007) Arch. Biochem. Biophys. 464:19-27 and He et al. (2002) Oncogene 21:2623-2633. Apoptosis is also detected biochemically by the activation of caspases 3, 8 and 9 using anti-human pro-caspase antibodies and Western blotting as described in He et al. (2002) Oncogene 21:2623-2633. These morphological and biochemical changes occur several hours after changes in the cytoplasmic Na⁺ and ER Ca²⁺ levels are observed and coincide with growth inhibition of the tumor cells detected by the modified MTT assay described in Mossman (1983) J. Immunol. Methods 65:55-63. These basic changes and the sequence of events occur in all tumor cell lines overexpressing MAT-8. The specific sites of chemical modification by AS/HNO are assayed using adenovirus-mediated expression of cysteine-free MAT-8 in cultured prostate and breast cancer cells. Cells expressing cysteine-free MAT-8 (MAT-8(—C)) show no growth inhibition and thus are insensitive to AS.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A method for inhibiting MAT-8 protein function in a neoplastic cell overexpressing MAT-8 protein, which method comprises administering an effective amount of a nitroxyl-donating compound to said cell thereby inhibiting the MAT-8 protein function.

2. The method of claim 1, wherein the MAT-8 protein function is the inhibition of Na⁺/K⁺ transport across a plasma membrane by a Na⁺/K⁺ pump.

3. The method of claim 1, wherein the nitroxyl-donating compound is Angeli's salt of the formula (Na₂N₂O₃).

4. The method of claim 1, wherein the neoplastic cell is selected from the group consisting of a neoplastic prostate cell, a neoplastic breast cell, a neoplastic pancreatic cell, a neoplastic colon cell and a neoplastic lung cell.

5. The method of claim 4, wherein the neoplastic prostate cell is androgen dependent.

6. The method of claim 4, wherein the neoplastic prostate cell is androgen independent.

7. The method of claim 4, wherein the neoplastic breast cell is estrogen or progesterone dependent.

8. The method of claim 4, wherein the neoplastic breast cell is estrogen or progesterone independent.

9. A method for treating a neoplastic prostate cell overexpressing MAT-8, which method comprises administering an effective amount of a nitroxyl-donating compound to said cell.

10. The method of claim 9, wherein the nitroxyl-donating compound is Angeli's salt of the formula (Na₂N₂O₃).

11. The method of claim 9, wherein the cell is androgen dependent.

12. The method of claim 9, wherein the cell is androgen independent.

13. A method for treating a neoplastic breast cell overexpressing MAT-8 protein, which method comprises administering an effective amount of a nitroxyl to said cell.

14. The method of claim 13, wherein the nitroxyl-donating compound is Angeli's salt of the formula (Na₂N₂O₃).

15. The method of claim 13, wherein the cell is estrogen or progesterone dependent.

16. The method of claim 13, wherein the cell is estrogen or progesterone independent.

17. A method for treating a patient suffering from a cancer overexpressing MAT-8 protein, the method comprises administering a therapeutically effective amount of a nitroxyl-donating compound and wherein the amount is effective to inhibit MAT-8 protein function thereby treating the patient.

18. The method of claim 17, wherein the cancer is selected from the group consisting of prostate cancer, breast cancer, pancreatic cancer, colon cancer and lung cancer.

19. The method of claim 18, wherein the cancer is susceptible to hormone therapy.

20. The method of claim 18, wherein the cancer is not susceptible to hormone therapy.

21. The method of claim 17, wherein the nitroxyl-donating compound is Angeli's salt of the formula (Na₂N₂O₃).

22. A method for treating a patient suffering from a prostate cancer overexpressing MAT-8 protein, the method comprises administering a therapeutically effective amount of a nitroxyl-donating compound and wherein the amount is effective to inhibit MAT-8 protein function thereby treating the patient.

23. The method of claim 22, wherein the cancer is susceptible to hormone therapy.

24. The method of claim 22, wherein the cancer is not susceptible to hormone therapy.

25. The method of claim 22, wherein the nitroxyl-donating compound is Angeli's salt of the formula (Na₂N₂O₃).

26. A method for treating a patient suffering from a breast cancer overexpressing MAT-8 protein, the method comprises administering a therapeutically effective amount of a nitroxyl-donating compound and wherein the amount is effective to inhibit MAT-8 protein function thereby treating the patient.

27. The method of claim 26, wherein the cancer is susceptible to hormone therapy.

28. The method of claim 26, wherein the cancer is not susceptible to hormone therapy.

29. The method of claim 26, wherein the nitroxyl-donating compound is Angeli's salt of the formula (Na₂N₂O₃).
